SEQUENCE LISTING (9 sequences)

SEQ ID NO: 1 - DNA, 5430 bp, Lactuca sativa
  gDNA
atggcgtcttcttcaagttcatccaatcaaaagagcttcaagtatcaggtgttcttaagctttacaggagaagacacacgtaagaccttcgttgatcatctttatgcttctcttaagcaaaaaggtattcataccttcagggataacgaggagctggagaaaggaaaacggatcgatgagctcttcaaagccattgaagagtcgaggttctttatcatagttttctccaaaaactatgcatcttcttcttggtgcttgaaggaggtcacgaagattatggaatgccaagatgggaaccaacagattgcttatccactcttttatggtgtggagcccagcgacatccgccatcaaactgggccagttggaagagcaatcgccaaacataaggacaacgaacaaattaagaaatgggaaaaagctctggaagatgcaggcaatctggttgggtgggaccttaaaaacattgctaatgggtataaattccttccttcatcatatctctgcaaaaaatttctttagtttttgtattatcaatctaggtccagccaacgatgtagatgggtgatcaatattatattgtatgtctctaacacaaagtttggtcttttatgtgatgatagaacctccttaaagtcagcaacaacttcaccagatctgacttttcttttctttggtgtcggggtatttcttccttgatttatgaagtatggattagatttgtagttagtatttaagttttagaaacaatatgcttataaatactaactattattgaaatcgtttccttcaaattaaagaggaatatttgcatttttgcattgatctttggtgtctgggtatttctacagcaaaaattgtttttttttctttttcagttgagccttataaacaaagcatcaaacattaatcttcttatttccaacattctatatatatatatatatatttgtgaaacatgaaatatgtgtatggtatagtgctgaaactaattgatcatgtgggatcttgagcaagctacttaaatattatttaatctacaggcatgaagctgaagccatcacaaaaattattgaagaaatttcattgaagctaggttccattcatttgggcaatgatgaacatctgataggcatggaacggcggatgcaagccctggagccgtctttggggatcggattgaacgataaagcccggatgatagggatcaaggggatgggaggcattgggaagacgactttagccaaagctatttttaataaattttcttcccattttgaaggtagtagctttgttgctgacataagagaagtttcaaaaaagaagggcttggagtcattgcaacaacaaatcctttcggatgttcttaaagatgaacgcatgcgtgtgggaagtgttaatgaaggggaaagaatcatgagaatgaggctaccgtataagaaagtgcttcttgttctagacgatgtggatgatacaaaacagcttgaggcgttagctggtgattggtttaagaatggaagtagaatcatcattacaacaagagacgagaatgtgctgctatcacatgaagtgaaaaagaaatggattcatgatgtcgatttcttgtcggatgaggaagcaattcgcctcttcctttggtttgcgtttaggagatatattccagatcaagggtacgaagagctcacagcccgagtagtacgttatgctgctggtcttcccttaaagattagagttttgggttcccatctgtgtggtgaaaataaggatgtatggagagatgcacttaaaagacttgaaaaaattccactacaagagactctagatgtattggaaataagctataacagcctagaagatgatcacaaggaaatcttcttagatgttgcatgcttcttgaacgggctttctcatgaatttgcaattagaatccttgagagctgtggatttcatgctacatatggtttaagaattcttgagcacaaatctcttgtaacaatttcagatggtagattgggcatgcatgatacaatacaagaattgggcaagaatattattcggcgagagcacccccgtgaactcaacaaacatagccgactctggaatacagaggaaattgtagaattattgtctgatgatgaggtaaggatcgaatttattgcaagcaacatatttttaattataggtactgcagcctctgacacaagtttcatggtgactgctttttggatcatatatagggtactgcaacctcaacatgtaccggaatgctgctgctcttgctgctgtgtgaactgagtcctgaaattatcatccaaaaacttggaaacctgaagagacttagatatctttgtgttttggggatcgttagtgactgtttccctagtgactggaagtttgatgaaacgaaaccctcctttccgaattcgttacagtatctgatatgggataaataccctggtttctctttaccccacacatttcgagcaaagaatcttgttggacttgaattgactggaagcagaatcacgcaactatgggaaagtagtgaaagaaaggtacattggttgattcaatgtaatgtttttcgtctatttgttggtcgatacatcaatactctgtctaacttttgttctttttgcatatgatataatctcttaagaagctcaagttcttcctcctgggtaattcaaacttgaggacccttcgggaaagtggggaaagaatggtacagtggttgattcagtgtaatgctttcgtccacaacaatactctgtctaatttttttcttctttttgcatttgataggttcttgagaaactcaggttcctgaaccttaaaaattcgaaggtgagcatccttgaccttgggatgactcccaatcttgagaggttagatcttgaaggatgtcatgatttgaaagagatttatgcgcctgctggatgtctaaaaaggcttatctacatagatttacgtggttgcccatggtctgtatcttttccttttgttaaacagttggaaccacatgtgcttcttagtctacctgtgttagctgtgaaagtggatcccatggaggcatttccaagggactccacaaacaatctacgatttacatttgtgtattataaagaactaccctcgtcaagagaaggaattaattttaagagtgtttttctagatcttcagccttgcacaaaacttgagagcgtttcaggaagcatttgtggcttacaacatctaagagaagttaaattccagggctgtattccggaggtgcccaaggacattgaccagctgaaaggcttgcaacaactcattttgttatctacacacgtcaaacgtcttcctgatagcgtttgtatgttgaaacatctgaaatctctcaaacttagtgattgccagcatcttgaagagttgccggagaatcttggtttgttagaaaatttagaggagctgagtttgtcgactgcaagtattaggcgtcttcctgatagtgtttgtatgttgaaacatctgaaatctctcaaacttggagattgccaacatcttgaagagttaccggagaaccttggctggttaggaaatttagaggagctgatgttcatttctataagtattagacgtcttccagatagcatttgcatgctgagtcatctgaaatctctacaccttgactcttgtggacgcctcgagaagttacccgaggatattgggcaactaaagagtttagagatgctaaacctgggacagtgtgaatctttacgagatattccaaacagcatctgtaacatgaaatctttgactcatctctatctccgcgaatgtagacaagttgagaaattgccagaggatttgggaaacctaaagcttttacaagggttaaatatagggtttacaggaataagtcatcttccacatagtatttcctcgatgaatggtctacttgttttcggatccatatcacttcttcagtcttgtgcttttgcaaccgagatatatacccatgaagtacttggaccccactgccgcatacaagccatggatagcacactggcacacacagaattaaggagccagtccgaacagaagtatcttaccaggtatttcgttcttttttttattaattttcttatatactttagtaaatcatggagtggtgtggaatattaatcaggctgtgcatgggttggttttgaaaaatttcggttttacttcggtttcggttttactttggatgcagaagagctaaacctagactcctatatactgtttaaaatataatatgaatacaagaccaaccaaaaccaaacatgaccatgtcctgattccagattcctttatgtactgatgatacagggagggcaaggaaatattaattgaagatggtgatgagggtacactaggaggaggtattttgtgtttttttttcaagtcacgttttttattgaccaacatgtatgtatgcttttgtcacatcttgtatgtatttcagaatcttctgatgatgttaataaacgaatgagagtgagccacatgggaaccaactcagagtatctgcagaacaaagaactcatcaggtttgtgtacatttcttacacatgacatgacatgttgtatagtatttgagaagtaatctagtgtttggtatggtattttgttggtttattgaatagattcttcgagaacctcatgtctgcagccgatccagaagaattcctaaaaatgctccaagtgatcacctccttacaaggcgacaacaatggttcttcagaaatgagtgaaaaggaaactgacatgattattaagatgctagcagaagagctccaaaacaggtctgaaatggcatcatctttcaacacccaaaatctagtggaaacaaataggagtaatattccacaagggcaacaaagtcaaaggagtggtacttctcaaggtcaagggtttttgaatcgtcagcccagctttcctaatattcaaagccaattgaggaatccagacatgcgtgaggtatatatgctattgtataatcaaaaagcatattaacagattgtgtatcaattgacatttgattgactgatgtttggtttattgattggaaagatgacacctgacatcaaattctctcgggaggatgcagaaagagcccaacaagtcatgtcctctttgtcaccagagaccatccatagactggtaaacactatttatatattcatgcattcattttttcttgatttttggattttggattttgttttattaaagatgtacgtggtggggttgttacagataaagttggctgatctaataaaaacagcatgtgaaggtgctgtaaaaaccaaaaattggcttctggggaggcaggggtttgttatggtcgtatgtatgcttctttttgcaatctttctccattggcttggattcattggcaattag

SEQ ID NO: 2 - DNA, 3957 bp, Lactuca sativa
  cDNA
atggcgtcttcttcaagttcatccaatcaaaagagcttcaagtatcaggtgttcttaagctttacaggagaagacacacgtaagaccttcgttgatcatctttatgcttctcttaagcaaaaaggtattcataccttcagggataacgaggagctggagaaaggaaaacggatcgatgagctcttcaaagccattgaagagtcgaggttctttatcatagttttctccaaaaactatgcatcttcttcttggtgcttgaaggaggtcacgaagattatggaatgccaagatgggaaccaacagattgcttatccactcttttatggtgtggagcccagcgacatccgccatcaaactgggccagttggaagagcaatcgccaaacataaggacaacgaacaaattaagaaatgggaaaaagctctggaagatgcaggcaatctggttgggtgggaccttaaaaacattgctaatgggcatgaagctgaagccatcacaaaaattattgaagaaatttcattgaagctaggttccattcatttgggcaatgatgaacatctgataggcatggaacggcggatgcaagccctggagccgtctttggggatcggattgaacgataaagcccggatgatagggatcaaggggatgggaggcattgggaagacgactttagccaaagctatttttaataaattttcttcccattttgaaggtagtagctttgttgctgacataagagaagtttcaaaaaagaagggcttggagtcattgcaacaacaaatcctttcggatgttcttaaagatgaacgcatgcgtgtgggaagtgttaatgaaggggaaagaatcatgagaatgaggctaccgtataagaaagtgcttcttgttctagacgatgtggatgatacaaaacagcttgaggcgttagctggtgattggtttaagaatggaagtagaatcatcattacaacaagagacgagaatgtgctgctatcacatgaagtgaaaaagaaatggattcatgatgtcgatttcttgtcggatgaggaagcaattcgcctcttcctttggtttgcgtttaggagatatattccagatcaagggtacgaagagctcacagcccgagtagtacgttatgctgctggtcttcccttaaagattagagttttgggttcccatctgtgtggtgaaaataaggatgtatggagagatgcacttaaaagacttgaaaaaattccactacaagagactctagatgtattggaaataagctataacagcctagaagatgatcacaaggaaatcttcttagatgttgcatgcttcttgaacgggctttctcatgaatttgcaattagaatccttgagagctgtggatttcatgctacatatggtttaagaattcttgagcacaaatctcttgtaacaatttcagatggtagattgggcatgcatgatacaatacaagaattgggcaagaatattattcggcgagagcacccccgtgaactcaacaaacatagccgactctggaatacagaggaaattgtagaattattgtctgatgatgagggtactgcaacctcaacatgtaccggaatgctgctgctcttgctgctgtgtgaactgagtcctgaaattatcatccaaaaacttggaaacctgaagagacttagatatctttgtgttttggggatcgttagtgactgtttccctagtgactggaagtttgatgaaacgaaaccctcctttccgaattcgttacagtatctgatatgggataaataccctggtttctctttaccccacacatttcgagcaaagaatcttgttggacttgaattgactggaagcagaatcacgcaactatgggaaagtagtgaaagaaagaagctcaagttcttcctcctgggtaattcaaacttgaggacccttcgggaaagtggggaaagaatggttcttgagaaactcaggttcctgaaccttaaaaattcgaaggtgagcatccttgaccttgggatgactcccaatcttgagaggttagatcttgaaggatgtcatgatttgaaagagatttatgcgcctgctggatgtctaaaaaggcttatctacatagatttacgtggttgcccatggtctgtatcttttccttttgttaaacagttggaaccacatgtgcttcttagtctacctgtgttagctgtgaaagtggatcccatggaggcatttccaagggactccacaaacaatctacgatttacatttgtgtattataaagaactaccctcgtcaagagaaggaattaattttaagagtgtttttctagatcttcagccttgcacaaaacttgagagcgtttcaggaagcatttgtggcttacaacatctaagagaagttaaattccagggctgtattccggaggtgcccaaggacattgaccagctgaaaggcttgcaacaactcattttgttatctacacacgtcaaacgtcttcctgatagcgtttgtatgttgaaacatctgaaatctctcaaacttagtgattgccagcatcttgaagagttgccggagaatcttggtttgttagaaaatttagaggagctgagtttgtcgactgcaagtattaggcgtcttcctgatagtgtttgtatgttgaaacatctgaaatctctcaaacttggagattgccaacatcttgaagagttaccggagaaccttggctggttaggaaatttagaggagctgatgttcatttctataagtattagacgtcttccagatagcatttgcatgctgagtcatctgaaatctctacaccttgactcttgtggacgcctcgagaagttacccgaggatattgggcaactaaagagtttagagatgctaaacctgggacagtgtgaatctttacgagatattccaaacagcatctgtaacatgaaatctttgactcatctctatctccgcgaatgtagacaagttgagaaattgccagaggatttgggaaacctaaagcttttacaagggttaaatatagggtttacaggaataagtcatcttccacatagtatttcctcgatgaatggtctacttgttttcggatccatatcacttcttcagtcttgtgcttttgcaaccgagatatatacccatgaagtacttggaccccactgccgcatacaagccatggatagcacactggcacacacagaattaaggagccagtccgaacagaagtatcttaccagggagggcaaggaaatattaattgaagatggtgatgagggtacactaggaggagaatcttctgatgatgttaataaacgaatgagagtgagccacatgggaaccaactcagagtatctgcagaacaaagaactcatcagattcttcgagaacctcatgtctgcagccgatccagaagaattcctaaaaatgctccaagtgatcacctccttacaaggcgacaacaatggttcttcagaaatgagtgaaaaggaaactgacatgattattaagatgctagcagaagagctccaaaacaggtctgaaatggcatcatctttcaacacccaaaatctagtggaaacaaataggagtaatattccacaagggcaacaaagtcaaaggagtggtacttctcaaggtcaagggtttttgaatcgtcagcccagctttcctaatattcaaagccaattgaggaatccagacatgcgtgagatgacacctgacatcaaattctctcgggaggatgcagaaagagcccaacaagtcatgtcctctttgtcaccagagaccatccatagactgataaagttggctgatctaataaaaacagcatgtgaaggtgctgtaaaaaccaaaaattggcttctggggaggcaggggtttgttatggtcgtatgtatgcttctttttgcaatctttctccattggcttggattcattggcaattag

SEQ ID NO: 3 - protein, 1318 aa, Lactuca sativa
  protein sequence
MASSSSSSNQKSFKYQVFLSFTGEDTRKTFVDHLYASLKQKGIHTFRDNEELEKGKRIDELFKAIEESRFFIIVFSKNYASSSWCLKEVTKIMECQDGNQQIAYPLFYGVEPSDIRHQTGPVGRAIAKHKDNEQIKKWEKALEDAGNLVGWDLKNIANGHEAEAITKIIEEISLKLGSIHLGNDEHLIGMERRMQALEPSLGIGLNDKARMIGIKGMGGIGKTTLAKAIFNKFSSHFEGSSFVADIREVSKKKGLESLQQQILSDVLKDERMRVGSVNEGERIMRMRLPYKKVLLVLDDVDDTKQLEALAGDWFKNGSRIIITTRDENVLLSHEVKKKWIHDVDFLSDEEAIRLFLWFAFRRYIPDQGYEELTARVVRYAAGLPLKIRVLGSHLCGENKDVWRDALKRLEKIPLQETLDVLEISYNSLEDDHKEIFLDVACFLNGLSHEFAIRILESCGFHATYGLRILEHKSLVTISDGRLGMHDTIQELGKNIIRREHPRELNKHSRLWNTEEIVELLSDDEGTATSTCTGMLLLLLLCELSPEIIIQKLGNLKRLRYLCVLGIVSDCFPSDWKFDETKPSFPNSLQYLIWDKYPGFSLPHTFRAKNLVGLELTGSRITQLWESSERKKLKFFLLGNSNLRTLRESGERMVLEKLRFLNLKNSKVSILDLGMTPNLERLDLEGCHDLKEIYAPAGCLKRLIYIDLRGCPWSVSFPFVKQLEPHVLLSLPVLAVKVDPMEAFPRDSTNNLRFTFVYYKELPSSREGINFKSVFLDLQPCTKLESVSGSICGLQHLREVKFQGCIPEVPKDIDQLKGLQQLILLSTHVKRLPDSVCMLKHLKSLKLSDCQHLEELPENLGLLENLEELSLSTASIRRLPDSVCMLKHLKSLKLGDCQHLEELPENLGWLGNLEELMFISISIRRLPDSICMLSHLKSLHLDSCGRLEKLPEDIGQLKSLEMLNLGQCESLRDIPNSICNMKSLTHLYLRECRQVEKLPEDLGNLKLLQGLNIGFTGISHLPHSISSMNGLLVFGSISLLQSCAFATEIYTHEVLGPHCRIQAMDSTLAHTELRSQSEQKYLTREGKEILIEDGDEGTLGGESSDDVNKRMRVSHMGTNSEYLQNKELIRFFENLMSAADPEEFLKMLQVITSLQGDNNGSSEMSEKETDMIIKMLAEELQNRSEMASSFNTQNLVETNRSNIPQGQQSQRSGTSQGQGFLNRQPSFPNIQSQLRNPDMREMTPDIKFSREDAERAQQVMSSLSPETIHRLIKLADLIKTACEGAVKTKNWLLGRQGFVMVVCMLLFAIFLHWLGFIGN

SEQ ID NO: 4 - DNA, 121 bp, Artificial Sequence
  Marker 1
cagcaccaccacccccaaacaccaacaatggtgtcttcttctccggcacaggccatctttcaccttacactgttacttatcatatcttcatgtatacctccttccttttcccaaagcactc

SEQ ID NO: 5 - DNA, 141 bp, Artificial Sequence
  Marker 2
gtggtgtgtctggttttgactttggtgttgacccgaatcttgatcctgagctggcacttgctcttcgtgtctccatggaagaagaaagagcaaggcaagaagctgctgctaaaaaagctgcagatgattcttcaaagaatg

SEQ ID NO: 6 - DNA, 121 bp, Artificial Sequence
  Marker 3
cagcaccaccacccccaaacaccaacaatggtgtcttcttctccggcacaggccatcttttaccttacactgttacttatcatatcttcatgtatacctccttccttttcccaaagcactc

SEQ ID NO: 7 - DNA, 141 bp, Artificial Sequence
  Marker 4
gtggtgtgtctggttttgactttggtgttgacccgaatcttgatcctgagctggcacttgctcttcgtgtttccatggaagaagaaagagcaaggcaagaagctgctgctaaaaaagctgcagatgattcttcaaagaatg

SEQ ID NO: 8 - DNA, 240 bp, Artificial Sequence
  VIGSc
gaccagttgaaaagcttggaacaactcattttgttgtctacacacgtcaaacgtcttcctgatagtgtttgtatgttgaaacatttgaaatctctcaacattagtgattgccagcatcttgaagagttgccggagaatcttggtttgttagaaaatttaatggagctgagtttatcgactacaagtattcagcgtcttcctgatagtgtttgtatgttgaaaaatctgaaatctctcgaa

SEQ ID NO: 9 - DNA, 240 bp, Artificial Sequence
  VIGSb
ttagctgtgaaagtggatcccttggaggattttccaaaggaccacacaaacaatctacgatttacatttgtatattataaagaaccacctttgtcaagagaaggaattgattataagactgtttttctagatcttcagccttgcacaaaacttgagagtgtttcaggaagcatttgtggcttacaacatctaagagaagttaaatttcagggctgtattacggaggttcctaacgacatt

(19) United States
(12) Patent Application Publication
(10) Pub. No.: US 2025/0031646 A1
(43) Pub. Date: Jan. 30, 2025

(54) LETTUCE PLANT RESISTANT TO DOWNY MILDEW AND RESISTANCE GENE

(71) Applicant: ENZAZADEN BEHEER B.V., Enkhuizen (NL)

(72) Inventors: Bas Ter Riet, Medemblik (NL); Mathieu André Pel, Enkhuizen (NL); Maarten Eduard Rouwet, Enkhuizen (NL); IIJA ROOBEEK, Dirkshom (NL)

(21) Appl. No.: 18/722,389

(22) PCT Filed: Apr. 5, 2022
(86) PCT No.: PCT/EP22/59008

§ 371 Date: Jun. 20, 2024

(30) Foreign Application Priority Data
Dec. 21, 2021 (WO) PCT/EP2021/087080

(51) Int. Cl. A01H 6/14; A01H 1/00; A01H 5/10

(52) U.S. Cl. CPC A01H 6/1472; A01H 1/1255; A01H 5/10

(57) ABSTRACT

The present invention relates to a lettuce plant that is resistant to downy mildew, more specifically to a lettuce plant that comprises a resistance gene that confers broad spectrum resistance to oomycetes in lettuce, more specifically B. lactucae. Furthermore, the present invention relates to a resistance gene and a method for obtaining a lettuce plant that is resistant to downy mildew, wherein the method comprises the step of introducing said resistance gene into a lettuce plant.

The present invention relates to a lettuce plant that is resistant to downy mildew, more specifically to a lettuce plant that comprises a resistance gene that confers broad spectrum resistance to oomycetes in lettuce, more specifically Bremia lactucae. Furthermore, the present invention relates to a resistance gene and a method for obtaining a lettuce plant that is resistant to downy mildew, wherein the method comprises the step of introducing said resistance gene into a lettuce plant.

Downy mildew refers to several types of oomycete microbes that are pathogens of plants. Downy mildew can originate from various species, but mainly of Peronospora, Plasmopara and Bremia. Downy mildew is a problem in many food crops, for example in lettuce caused by B. lactucae, affecting the production of this crop worldwide. Plants that are being affected include food crops such as brassicas (e.g. cabbage), grape, spinach, lettuce, onion, and cucumber. Downy mildew infection shows symptoms of discoloured areas on upper leaf surfaces in combination with white, grey or purple mould located on the lower side of the leaf facing the floor. Disease is spread from plant to plant by airborne spores.

Lettuce, mostly known as Lactuca sativa, but also including Lactuca species such as L. serriola. L. saligna or L. virosa, is a very important crop worldwide. Some of the most popular varieties available are Iceberg, Romaine, Butterhead, Batavia and Oakleaf. There are many plant pathogens that affect L. sativa, and some of the diseases caused by these pathogens are downy mildew, sclerotinia rot, powdery mildew, fusarium wilt of which the most important disease is lettuce downy mildew, which is caused by the B. lactucae, an oomycete pathogen that belong to Peronosporaceae.

For some vegetable crops, such as lettuce, cultivars with resistance to downy mildew are available. However, the pathogen under pressure will mutate to break down the disease resistance and new disease resistance in crops is needed to control infection. Especially in lettuce the occurrence of downy mildew resistance is particularly complex as there are many different races, and new downy mildew resistant species emerging all the time, as found in European and the USA markets.

In lettuce, infection of B. lactucae result in yellow to pale green lesions that eventually become necrotic due to secondary pathogens leading to major crop losses. Fungicides can be used to control B. lactucae, but eventually B. lactucae becomes immune to these chemicals, because over time the pathogen also acquires resistance to fungicides. Furthermore, there are multiple lettuce varieties available that are resistant to B. lactucae but resistance is quickly overcome because new Bremia races develop rapidly. Therefore, it is of the utmost importance to find other methods to control B. lactucae infection. Most preferably is to identify a resistance gene that gives broad resistance against B. lactucae and to provide for lettuce plants that are resistant to downy mildew. Therefore, identification of resistance genes is a promising alternative.

During the development of new disease or pathogen resistant plants often traits are being combined, for example by introgression of a genetic locus comprising one or more resistance gene, thereby combining multiple resistance genes to combat the pathogen being able to overcome the resistance. However the introgression fragments often comprise, apart from the resistance gene of interest, other genetic elements that may negatively affect the plant in terms of yield, growth, vitality, and seed production. For example introgression of (additional) new resistance genes in lettuce often result in a severe reduction in seed production as a result of linkage drag. Therefore, apart from the generation of improved disease resistance in plant, there is also great benefit to reduce this so called linkage drag which becomes an increasing hurdle in plant breeding. Single event introgression as well as marker-aided selection techniques in the flanking regions of the resistance gene to reduce the introgression segment can play important role here, with the objective of minimizing residual genetics (apart from the resistance gene) being transferred to the parent plant and to eliminate linkage drag effects. Gene editing and the use of sequence information in genome-wide selection will further add to the precision of reduction of linkage drag.

Considering the above, there is a need in the art to provide plants that are resistant to downy mildew and wherein plants have a broad-spectrum resistance against this pathogen. Furthermore, it is an object of present invention to provide plants having a broad-spectrum downy mildew resistance, while maintaining optimal seed production of said downy mildew resistant plant and to provide a method to obtain such downy mildew resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a downy mildew resistant lettuce plant, wherein said lettuce plant comprises a V06 resistance gene encoding a protein having at least 90%, preferably at least 95%, more preferably at least 98%, most preferably 100% sequence identity with amino acid sequence of SEQ ID No. 3 providing downy mildew resistance, wherein said lettuce plant is resistant to B. lactucae races Bl:16 to Bl:37. The downy mildew resistance conferring gene V06 is a dominant resistance trait, and may be homozygous or heterozygous present in a downy mildew resistant lettuce plant. The resistance gene against B. lactucae has been found on chromosome 8 in an area that had not been linked to plant disease resistance in lettuce. This V06 resistance gene of the present invention gives resistance to B. lactucae races Bl:16-37EU and also Bl:1-9US wherein said Bremia races have been characterized and classified according to the SEXTET code by IBEB (International Bremia Evaluation Board). Furthermore, previous disease resistance test show that the V06 resistance gene further provides resistance to the “older”, less recent Bremia races Bl:1 to Bl:15. Therefore the V06 resistance gene provides full spectrum resistance to Bl:1-37EU.

The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. There are three major subfamilies of plant NBS-LRR proteins defined by the Toll/interleukin-1 receptor (TIR) also called TNLs, the coiled-coil (CC) motifs in the amino-terminal domain containing NBS-LRRs also called CNLs and RPW8-NLTRs also called RNLs. A typical R gene contains an NB-ARC domain which is proposed to regulate activity of the R protein.

The V06 resistance gene is unique since it is an NBS-LRR gene with a transmembrane region on the C terminus. It is expected by prediction programs that the part upstream of this transmembrane domain is localized outside the cell. This gene is initially picked up by fine mapping and VIGS experiments based on the L. sativa genome. The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. In addition the V06 gene comprises several LRR regions within the gene that may be beneficial and contributes to generate a new R gene that confers broad spectrum Bremia resistance.

The presence of the V06 resistance gene will provide broad spectrum Bremia resistance to lettuce plants. To decrease the chances of the pathogen overcoming the resistance, as often seen with R genes, multiple R genes can be combined to enhance the durability of disease resistance. For example, the downy mildew resistant lettuce plant of the present invention may further comprise one or more resistance genes located on chromosome 8 at a significant distance from the V06 resistance gene or with R genes located at different linkage groups. V06 is outside the MRC8 cluster (major resistance cluster 8) and could therefore be used to combine it with an active Bremia resistance gene on MRC8. As such, stacking of multiple resistance genes will enable broad and durable Bremia resistance in lettuce.

To demonstrate that the V06 resistance gene provides Bremia resistance, this V06 resistance gene was silenced by tobacco rattle virus (TRV)-based virus-induced gene silencing (VIGS) to induce susceptibility to B. lactucae infection in resistant L. sativa lines containing the V06 resistance gene. With VIGS it was demonstrated that the V06 resistance gene was associated with downy mildew resistance, since VIGS induced gene silencing was used to create Bremia susceptibility in resistant Lactuca accessions containing only V06 resistance. Resistant lettuce plants were transiently transformed with a silencing construct specific against the V06 resistance gene which will result in the silencing of the resistance gene and as a consequence made the plant or plant organs susceptible to B. lactucae infection, thus by “removing” or silencing the V06 resistance gene via virus induced gene silencing.

According to another preferred embodiment, the present invention relates to the lettuce plant, wherein said V06 resistance gene comprises a coding sequence having at least 90%, preferably at least 95%, more preferably at least 98%, most preferably 100% sequence identity with SEQ ID No. 2. Said V06 resistance gene is preferably located on chromosome 8 of the lettuce plant.

According to yet another preferred embodiment, the present invention relates to the lettuce plant, wherein said plant comprises a genomic sequence having at least 90%, preferably at least 95%, more preferably at least 98%, most preferably 100% sequence identity sequence identity with SEQ ID No. 1.

According to yet another preferred embodiment, the present invention relates to the lettuce plant, wherein said plant does not comprise SEQ ID No. 6 and/or SEQ ID No. 7 and wherein said plant is capable of an average seed production of at least 5 g of seed per lettuce plant, preferably at least 9 g of seed per lettuce plant, more preferably at least 14 g of seed per lettuce plant. In a further preferred embodiment, the present invention relates to the lettuce plant, wherein the plant comprises SEQ ID No. 4 and SEQ ID No. 5: preferably herein said plant is capable of an average seed production of at least 5 g of seed per lettuce plant, preferably at least 9 g of seed per lettuce plant, more preferably at least 14 g of seed per lettuce plant. Introgression fragments often comprise, apart from the resistance gene of interest, other genetic elements that may negatively affect the plant in terms of yield, growth, vitality, and seed production. On average a susceptible lettuce plant, not comprising the V06 resistance gene of present invention, produces about 12 to 15 gram seed per plant. However the seed production capacity of the plant comprising the V06 resistance gene was affected due to genetic drag, resulting in a seed production of about less than 5 gram per plant. We were able to reduce the size of the introgression fragment comprising the V06 gene providing full spectrum Bremia resistance in lettuce, while at the same time maintaining seed production levels. When the Bremia resistant lettuce plant did not comprise marker SEQ ID No. 6 and/or SEQ ID No. 7, more preferably when the Bremia resistant lettuce plant comprised markers SEQ ID No. 4 and SEQ ID No. 5 (i.e. having a reduced V06 introgression fragment), the seed production was restored while at the same time the plant was resistant to Bremia. A Bremia resistant lettuce plant which does not comprise both markers SEQ ID No. 6 and SEQ ID No. 7 comprises the most reduced V06 introgression fragment, and therefore is expected to restore the seed production to wild type levels, i.e. to seed production levels of a lettuce plant which does not comprise the V06.

According to a preferred embodiment, the present invention relates to the lettuce plant, wherein the lettuce plant is further resistant to one or more of B. lactucae races selected from the group consisting of races Bl:1-15EU. Recent experiments show that a lettuce plant of the present invention comprising the V06 resistant gene is resistant to Bremia races from Bl:16 to Bl:37EU. Previous disease resistance tests on less recent Bremia races Bl:115EU (data not shown) show that the V06 resistance gene further provides resistance to these Bremia races. Resistance to B. lactucae in the lettuce of present invention comprises full spectrum resistance to B. lactucae races Bl:1 to Bl:37EU.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to a preferred embodiment, the present invention relates to the lettuce plant, wherein the V06 resistance gene is at least heterozygously present in the lettuce plant, preferably homozygously.

According to yet another preferred embodiment, the present invention relates to the lettuce plant, wherein the V06 resistance gene is obtainable, derived, or originates from a lettuce plant of L. virosa. Most preferably the present invention relates to the lettuce plant, wherein the V06 resistance gene is obtainable, derived, or originates from a lettuce plant deposited under number NCIMB 43827.

The present invention, according to a second aspect, relates to seed of a lettuce plant of the present invention, comprising a V06 resistance gene encoding a protein as described above. The seed comprises the V06 resistance gene as described above.

According to a preferred embodiment, the present invention relates to a resistance gene that confers resistance to B. lactucae in lettuce plants, wherein the resistance gene encodes for a protein that has at least 90%, preferably at least 95%, more preferably at least 98%, most preferably 100% sequence identity with SEQ ID No. 3. The V06 resistant gene is a dominant trait.

According to another preferred embodiment, the present invention relates to a resistance gene that confers resistance to B. lactucae in lettuce plants, wherein the coding sequence of said resistance gene has at least 90%, preferably at least 95%, more preferably at least 98%, most preferably 100% sequence identity with SEQ ID No. 2.

According to yet another preferred embodiment, the present invention relates to a resistance gene that confers resistance to B. lactucae in lettuce plants, wherein the resistance gene provides resistance to at least Bremia lactucae races Bl:16-37EU, preferably Bremia lactucae races Bl:1-37EU in lettuce. The resistance gene further provides resistance to B. lactucae US spectrum Bl:1-9US.

According to yet another preferred embodiment, the present invention relates to the resistance gene that confers resistance to B. lactucae in lettuce plants, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a method for identifying (i) a downy mildew resistant lettuce plant of the present invention or (ii) a seed from said plant wherein the method comprises the step of establishing, in the genome of a plant or seed the presence of a V06 resistance gene encoding a protein as defined above. The step of establishing, in the genome of the seed, the presence of any genetic information, including the presence of the V06 resistance gene encoding the protein as defined above, may suitably involve allowing the seed to grow into a plant and establishing the presence of the genetic information in the genome of the plant grown from the seed.

According to yet another preferred embodiment, the present invention relates to the method for identifying a downy mildew resistant lettuce plant of present invention, wherein the step of establishing, comprises establishing the presence of one or more sequences selected from the group consisting of SEQ ID No. 1, SEQ ID No. 2, SEQ ID No. 4, SEQ ID No. 5, SEQ ID No. 6, and SEQ ID No. 7, preferably SEQ ID No. 2 only, SEQ ID No. 2 in combination with SEQ ID No. 4, SEQ ID No. 2 in combination with SEQ ID No. 5, SEQ ID No. 2 in combination with SEQ ID No. 6, or SEQ ID No. 2 in combination with SEQ ID No. 7: most preferably the method comprises establishing the presence of SEQ ID No. 2 in combination with SEQ IDs No. 4 and 5. A downy mildew plant of present invention comprising the V06 resistance gene can be identified by the presence in the genome of said plant of one or more sequences selected from the group consisting of SEQ ID No. 1, SEQ ID No. 2, SEQ ID No. 4, SEQ ID No. 5, SEQ ID No. 6, SEQ ID No. 7, preferably only SEQ ID No. 2, or SEQ ID No. 2 and SEQ ID no. 4, or SEQ ID No. 2 and SEQ ID No. 5, or SEQ ID No. 2 and SEQ ID No. 6, or SEQ ID No. 2 and SEQ ID No. 7. Presence of the V06 is identified by SEQ ID No. 1 or 2. Most preferably the plant can be identified by the presence of SEQ ID No. 2 in combination with SEQ IDs No. 4 and 5. Furthermore, a reduction of the size of the introgression fragment comprising the V06 gene providing full spectrum Bremia resistance in lettuce can be identified, resulting in maintaining seed production levels. Such a Bremia resistant lettuce plant having improved seed production does not comprise marker SEQ ID No. 6 and/or SEQ ID No. 7, and/or the V06 resistance gene comprises SEQ ID No. 4 and/or SEQ ID No. 5 (i.e. having a reduced V06 introgression fragment).

The present invention, according to a further aspect, relates to a method for obtaining a lettuce plant that is resistant to downy mildew, wherein the method comprises the steps of,
a) crossing a lettuce plant comprised of the resistance gene of the present invention with a lettuce plant susceptible to downy mildew and which does not comprise said resistance gene,
b) optionally, selfing the plant obtained in step a) for at least one time,
c) selecting the plants that are resistant to downy mildew.

A plant having this resistant phenotype can be obtained via use of gene editing and/or mutation techniques, such as EMS mutagenesis or CRISPR/Cas in concert with cloning techniques on the V06 resistance gene to generate disease resistant crops. A resistance gene can be brought into the plant by known means including e.g. transgenic techniques or by introgression, wherein the resistance providing sequence(s) are introduced into the plant.

The present invention, according to a further aspect, relates to the use of a gene construct or plasmid for introducing a resistance gene into the genome of a plant or plant cell and providing broad spectrum resistance to downy mildew caused by one or more of B. lactucae races selected from the group of race Bl:1637EU wherein the gene construct is comprised of the resistance gene operably linked to expression providing sequences in said plant. The resistance gene of present invention may be transferred (e.g. by transformation or transfection) into plants, such as lettuce plants, using a plasmid or vector or linear gene construct that comprises the resistance gene of present invention. The V06 resistance gene, after being transferred into the lettuce plant would provide resistance to B. lactucae, i.e. resistance to at least B. lactucae of race Bl:16-37EU, preferably Bl:1-37EU.

The present invention will be further detailed in the following examples and figures wherein:

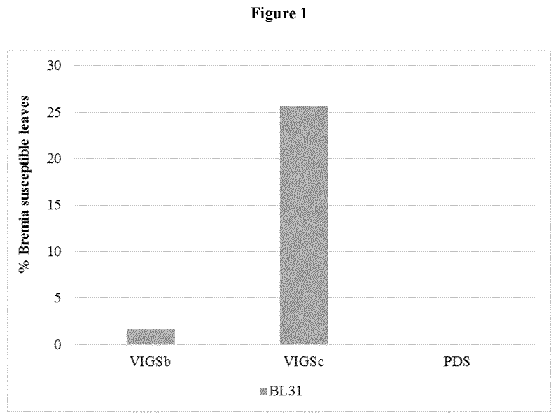

FIG. 1: shows the % of susceptible leaves of lettuce that have been infected with Bremia lactucae B131, after VIGS silencing of the V06 resistance gene of present invention of a lettuce plant of present invention comprising the V06 resistance gene using VIGS gene silencing constructs of Table 2 and subsequently infected with B. lactucae. As expected with transient gene silencing, VIGS gene silencing does not result in fully 100% silencing of the gene in all plants. However, the leaves from plants wherein the V06 resistance gene has been silenced by VIGS silencing (VIGSc silencing construct), showed a significant higher percentage of susceptible leaves when infected with Bremia as compared to plants where the V06 gene was not silenced (i.e. by VIGSb or PDS silencing construct on the plant comprising the V06 gene).

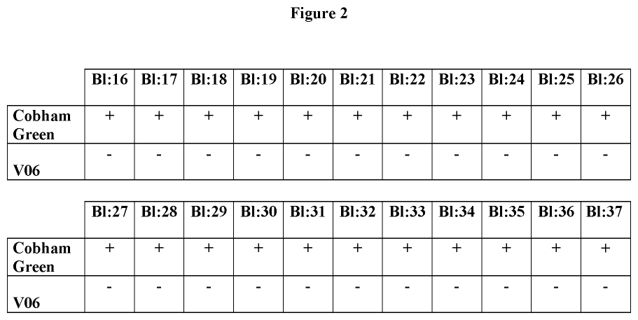

FIG. 2: shows an overview of the disease test performed with the most recent isolates of B. lactucae Bl:16-37EU on L. sativa lines Cobham Green, and the plant of present invention comprising the V06 resistance gene. The plant of present invention shows to be resistant to all tested downy mildew isolates, Bl:16-37EU, providing broad spectrum resistance.

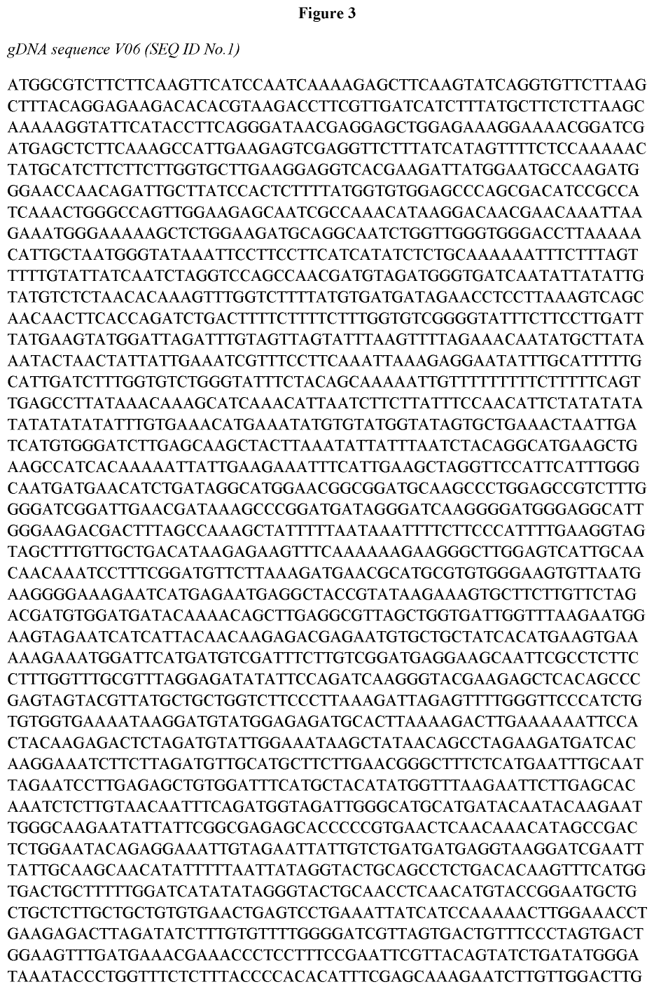

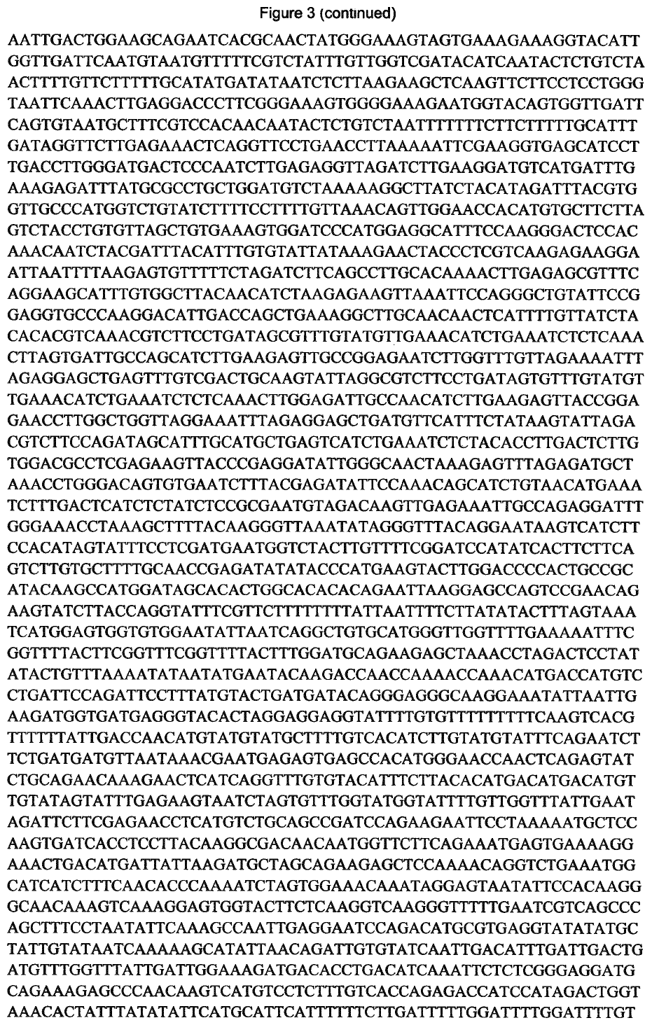

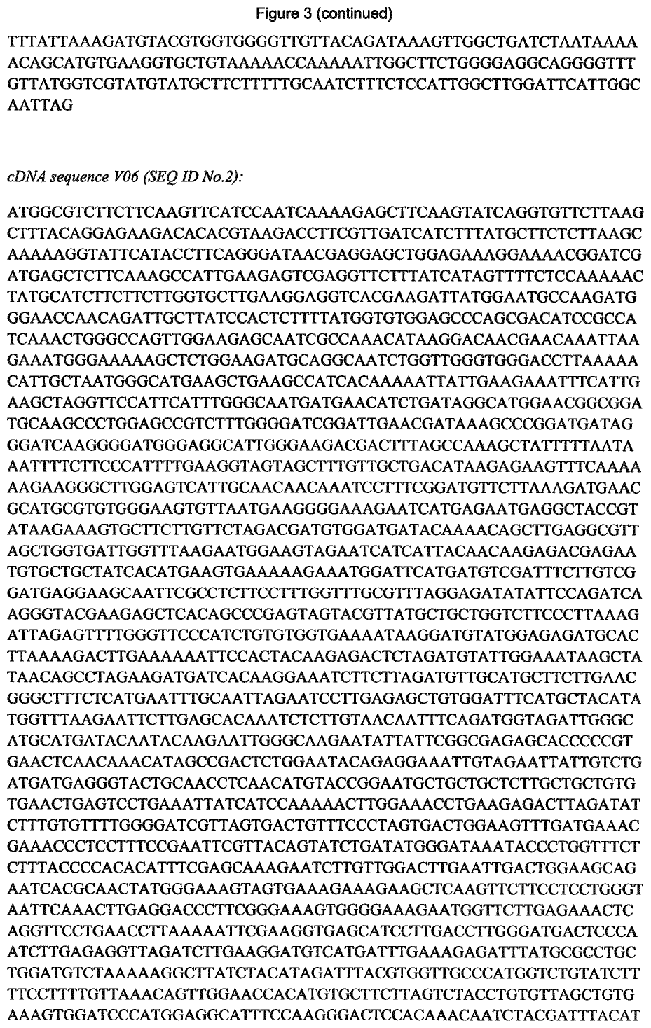

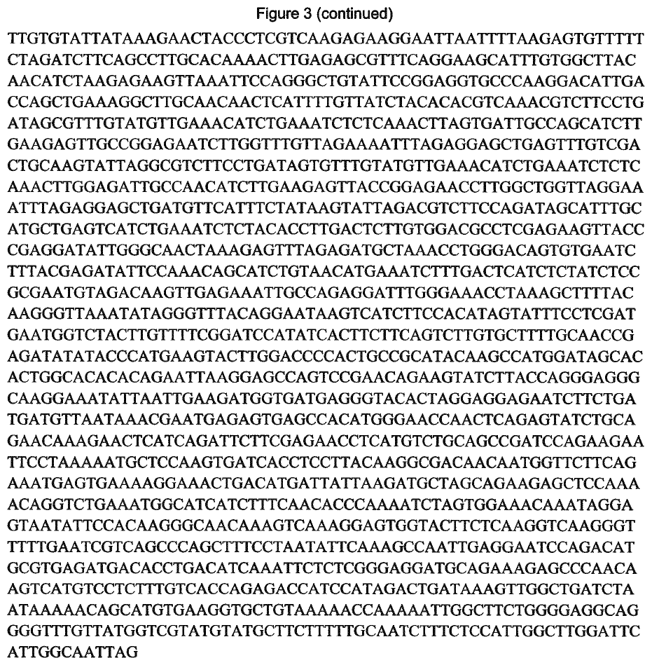

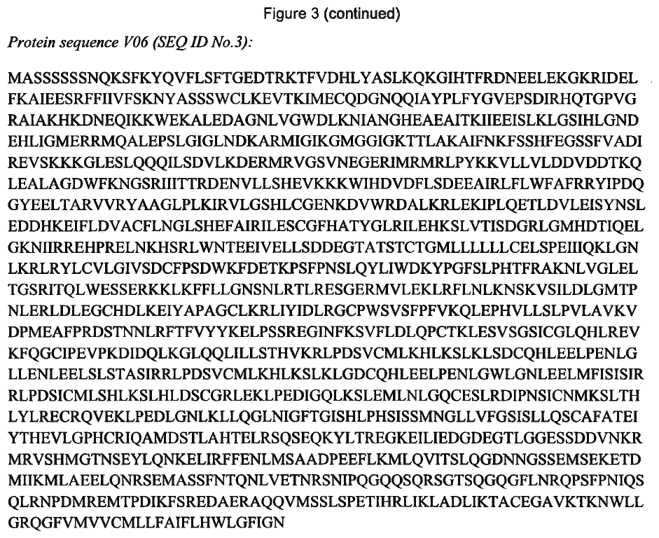

FIG. 3: shows the genomic (gDNA) sequence comprising the V06 resistance gene (SEQ ID No. 1), the coding (cDNA) sequence of the V06 resistance gene (SEQ ID No. 2) and its protein sequence (SEQ ID No. 3).

### EXAMPLES

Gene Mapping of V06 Resistance Gene in L. virosa

Gene mapping experiments were done to identify a resistance gene that is involved in full spectrum Bremia (B. lactucae) resistance in lettuce (L. sativa). The resistance gene was originally isolated from L. virosa and was mapped on chromosome 8. Furthermore, we were able to reduce the size of the introgression fragment comprising the V06 gene providing full spectrum Bremia resistance in lettuce, while at the same time maintaining seed production levels.

The identified resistance locus comprises two markers; the marker 3 (SEQ ID No. 6) and marker 4 (SEQ ID No. 7), providing a resistance locus which comprises a novel resistance gene identified as V06. Experiments on seed production levels show that when the resistance locus comprises marker 3 and/or marker 4, the seed production of the resistant plant is severely affected, due to the presence of linkage drag of the introgression fragment. After fine mapping in a population of about 12,000 plants there was one putative resistance gene present in the identified resistance locus.

TABLE 1
Marker sequences

| Marker | Sequence |
|---|---|
| Marker 1 (SEQ ID | CAGCACCACCACCCCCAAACACCAACAATGGTGTCTTCTTCTCC |
| No. 4) | GGCACAGGCCATCTTTCACCTTACACTGTTACTTATCATATCTTC |
| | ATGTATACCTCCTTCCTTTTCCCAAAGCACTC |
| Marker 2 (SEQ ID | GTGGTGTGTCTGGTTTTGACTTTGGTGTTGACCCGAATCTTGATC |
| No. 5) | CTGAGCTGGCACTTGCTCTTCGTGTCTCCATGGAAGAAGAAAGA |
| | GCAAGGCAAGAAGCTGCTGCTAAAAAAGCTGCAGATGATTCTTC |
| | AAAGAATG |
| Marker 3 (SEQ ID | CAGCACCACCACCCCCAAACACCAACAATGGTGTCTTCTTCTCC |
| No. 6) | GGCACAGGCCATCTTTTACCTTACACTGTTACTTATCATATCTTC |
| | ATGTATACCTCCTTCCTTTTCCCAAAGCACTC |
| Marker 4 (SEQ ID | GTGGTGTGTCTGGTTTTGACTTTGGTGTTGACCCGAATCTTGATC |
| No. 7) | CTGAGCTGGCACTTGCTCTTCGTGTTTCCATGGAAGAAGAAAGA |
| | GCAAGGCAAGAAGCTGCTGCTAAAAAAGCTGCAGATGATTCTTC |
| | AAAGAATG |

### Seed Production Levels of Plant Comprising V06 Resistance Gene

In a population of about 2,000 plants that homozygously comprise the V06 gene, the seed production levels were examined in order to improve seed production by reducing the introgression fragment comprising the V06 gene.

Lettuce plants of L. sativa are kept in a greenhouse at a temperature at daytime of about 21° C. and at night time at about 15° C., the average temperature in 24 h is about 19° C. Plants are grown under light conditions (about 6000 lux) for about 16 h per day. The lettuce plant is grown in a wet pot and it takes about 20 weeks from planting to harvesting of the seeds. Organic control is done every week against thrips and whitefly. After about 20 weeks the lettuce head comprising the seed is ripe and is harvested for seeds.

Experiments showed that when Bremia resistant lettuce plants comprise the locus comprising the V06 resistance gene (+V06 locus), seed production was severely affected producing less seeds per plant in comparison to plants (that are susceptible to Bremia) that do not comprise this V06 locus (−V06 locus), due to linkage drag of the V06 resistance locus. Experiments on seed production levels show that when the introgression fragment comprising the resistance locus is reduced, as indicated by the presence of marker 1 (SEQ ID No. 4) and/or marker 2 (SEQ ID No. 5), the seed production of the resistant plant is improved. Plants were therefore screened on a reduced size of the introgression fragment comprising the V06 gene, i.e. a fragment not comprising the marker 3 (VO6 short 3), or a fragment not comprising the marker 4 (V06 short 4), or a fragment that does not comprise both marker 3 and marker 4 (VO6 short 3 and 4).

Plants were screened on Bremia resistance (B129 and B131 were tested), while at the same time maintaining seed production levels (see Table 2). Results show that when the Bremia resistant plant did not comprise marker 3 or 4 (i.e. having a reduced V06 introgression fragment), the seed production was restored while at the same time the plant was resistant to Bremia. More specifically to Table 2:
in the variant designated “+V06 locus”, the introgression fragment comprised the V06 gene and markers 3 and 4;
in the V06 short 3 variant, the introgression fragment comprised the V06 gene and markers 1 and 4;
in the V06 short 4 variant, the introgression fragment comprised the V06 gene and markers 3 and 2;
in the V06 short 3 and 4 variant, the introgression fragment comprised the V06 gene and markers 1 and 2.

A Bremia resistant lettuce plant which does not comprise both markers SEQ ID No. 6 and SEQ ID No. 7 and/or comprises markers 1 and 2 (SEQ IDs 4 and 5, respectively) provided the most reduced V06 introgression fragment, and therefore is expected to restore the seed production to wild type levels, i.e. average weight of 15.7 g per plant. When combining the seed production levels of the plants missing either marker 3 or 4, the seed production levels are close to wild type production levels, as expected, i.e. to seed production levels of a lettuce plant having an average weight of 5.8+9.5=15.3 g per plant (*).

TABLE 2
Effect of V06 on seed production

| | Bremia resistance | Average weight of |
|---|---|---|
| Plant | (BI29/BI31) | seed/plant (g) |
| + V06 locus | +/+ | 1.9 |
| − V06 locus | −/− | 15.7 |
| + V06 short 3 | +/+ | 5.8 |
| + V06 short 4 | +/+ | 9.5 |
| + V06 short 3 and 4 | +/+ | 15.3* |

### V06 Resistance Gene Silencing Experiment Using Virus Induced Gene Silencing (VIGS)

To demonstrate that the V06 resistance gene provides Bremia resistance, the V06 resistance gene was silenced by tobacco rattle virus (TRV)-based virus-induced gene silencing (VIGS) to induce susceptibility to B. lactucae infection in L. sativa lines containing the V06 resistance gene. Tobacco rattle virus (TRV)-derived VIGS vectors have been abundantly described to study gene function in Arabidopsis thaliana, Nicotiana benthamiana, Solanum esculentum and other plants (see for example Huang C, Qian Y, Li Z, Zhou X.: Virus-induced gene silencing and its application in plant functional genomics. Sci China Life Sci. 2012: 55 (2): 99-108). With VIGS it was demonstrated that the V06 resistance gene was associated with downy mildew resistance, since VIGS induced gene silencing was used to create Bremia susceptibility in resistant Lactuca accessions comprising the V06 gene. Resistant lettuce plants were transiently transformed with a silencing construct specific against the resistance V06 gene which will result in the silencing of the resistance gene.

Briefly, lettuce plants containing the V06 resistance gene were silenced for V06 resistance gene by VIGS using different silencing construct to identify if this V06 resistance gene was indeed responsible for the observed resistance. Two VIGS-constructs were used, one (VIGSc) that results in specific silencing of the V06 resistance gene and VIGSb that silenced the intron of the V06 resistance gene. Furthermore, independent of resistance gene silencing the PDS gene is silenced as well that serves as positive control to indicate if VIGS is working and to determine the efficiency. The PDS gene is involved in carotenoid biosynthesis and is the first step in lycopene biosynthesis. This step is catalyzed by the enzyme phytoene desaturase (PDS). When silencing of the PDS gene is achieved, this results in bleached leaves. Experiments showed bleached leaves indicating that the VIGS silencing was achieved and performed correctly (data not shown). All plants that were VIGS inoculated were harvested and put in a tray and sprayed with Bremia to test the effect of the gene silencing on disease resistance.

The VIGS constructs were cloned in the K20 vector (See Table 3 for sequences, respectively SEQ ID No. 8, SEQ ID No. 9). The constructs were transformed and transiently expressed into a lettuce plant of present invention that is resistant to Bremia, using co-cultivation with agrobacterium (GV3101) to study the resistance gene function in relation to Bremia resistance. The % of susceptible Bremia leaves was observed in both groups and both silencing constructs. With the leaves of VIGS-experiments independent disease tests (see below) were performed to observe that when V06 resistance gene was silenced, plants became susceptible to Bremia.

Results (FIG. 1) indicate that when V06 was silenced by VIGS with the VIGSc construct the plants became susceptible after Bremia infection (B131) confirming that the resistance gene is linked to a resistance gene that provides the plant resistance against Bremia.

TABLE 3
VIGS constructs

| VIGS-constructs | Sequence |
|---|---|
| VIGSc | GACCAGTTGAAAAGCTTGGAACAACTCATTTTGTTGTCTACACA |
| (SEQ ID No. 8) | CGTCAAACGTCTTCCTGATAGTGTTTGTATGTTGAAACATTTGAA |
| | ATCTCTCAACATTAGTGATTGCCAGCATCTTGAAGAGTTGCCGG |
| | AGAATCTTGGTTTGTTAGAAAATTTAATGGAGCTGAGTTTATCG |
| | ACTACAAGTATTCAGCGTCTTCCTGATAGTGTTTGTATGTTGAAA |
| | AATCTGAAATCTCTCGAA |
| VIGSb | TTAGCTGTGAAAGTGGATCCCTTGGAGGATTTTCCAAAGGACCA |
| (SEQ ID No. 9) | CACAAACAATCTACGATTTACATTTGTATATTATAAAGAACCAC |
| | CTTTGTCAAGAGAAGGAATTGATTATAAGACTGTTTTTCTAGAT |
| | CTTCAGCCTTGCACAAAACTTGAGAGTGTTTCAGGAAGCATTTG |
| | TGGCTTACAACATCTAAGAGAAGTTAAATTTCAGGGCTGTATTA |
| | CGGAGGTTCCTAACGACATT |

### Disease Test and Biotest for Downy Mildew in Lettuce

Leaves of resistant plants transiently transformed with the above described VIGS constructs, were put in trays with moistened paperboard and infected with Bremia race 31. Infected seedlings are suspended in 20 mL water, filtered by cheesecloth and the flow-through is collected in a spray flask. The trays are spray-inoculated with the B. lactucae suspension. The trays are covered with a glass plate and stored in a climate chamber at 15° C. (12 hours of light). A black, opaque foil is placed over the trays for one day to improve growth of B. lactucae. After one day, the foil is removed. Experiments were performed in triple, and eight to ten days after infection leaves are phenotypically scored by eye on the presence of Bremia, i.e. being susceptible or resistant (FIG. 1).

Disease resistance tests show that the V06 resistance gene provides resistance to Bremia races from B1-37EU (See FIG. 2). Furthermore, disease resistance test show that the V06 resistance gene further provides resistance to US Bremia races Bl:1 to Bl:9 (results not shown). Previous disease resistance test have shown that the V06 resistance gene also provides resistance to Bl:1 to Bl:15 (results not shown), therefore the V06 resistance gene provides full spectrum resistance to Bl:1-37EU.

A single gene line comprising the V06 resistance gene was used internally to test Bremia diagnostic. Seeds of this line are deposited at NCIMB Ltd. Aberdeen. Scotland on 2 Aug. 2021 under the number NCIMB 43827.

1. A downy mildew resistant lettuce plant, wherein said lettuce plant comprises a V06 resistance gene encoding a protein having at least 90% sequence identity with amino acid sequence of SEQ ID NO: 3 providing downy mildew resistance, wherein said lettuce plant is resistant to Bremia lactucae races Bl:16-37EU and/or Bl:1-9US.

2. The lettuce plant according to claim 1, wherein said V06 resistance gene comprises a coding sequence having at least 90% sequence identity with SEQ ID NO: 2.

3. The lettuce plant according to claim 1, wherein said lettuce plant comprises a genomic sequence having at least 90% sequence identity with SEQ ID NO: 1.

4. The lettuce plant according to any one of the claim 1, wherein said lettuce plant does not comprise SEQ ID NO: 6 and/or SEQ ID NO: 7, and wherein said lettuce plant provides an average seed production of at least 5 g of seed per lettuce plant.

5. The lettuce plant according to claim 1, wherein the lettuce plant is further resistant to one or more of Bremia lactucae races selected from the group consisting of races Bl:1-15EU.

6. The lettuce plant according to claim 1, wherein the lettuce plant is selected from the group consisting of Lactuca sativa, Lactuca virosa, Lactuca saligna, Lactuca serriola, Lactuca aculeate, Lactuca georgica, Lactuca perennis, Lactuca tatarica, and Lactuca viminea.

7. The lettuce plant according to claim 1, wherein the V06 resistance gene is obtainable, derived, or originates from a lettuce plant deposited under number NCIMB 43827.

8. A seed of the lettuce plant according to claim 1.

9. A resistance gene that confers resistance to downy mildew in a lettuce plant, wherein the resistance gene encodes for a protein that has at least 90% sequence identity with SEQ ID NO: 3.

10. The resistance gene according to claim 9, wherein the coding sequence of said resistance gene has at least 90% sequence identity with SEQ ID NO: 2.

11. The resistance gene according to claim 9, wherein the resistance gene provides resistance to at least Bremia lactucae races Bl:16 to Bl:37 in the lettuce plant.

12. A method for identifying the downy mildew resistant lettuce plant according to claim or a seed of said lettuce plant, the method comprises the step of establishing presence of the V06 resistance gene in a genome of the lettuce plant or the seed.

13. The method according to claim 12,
wherein the step of establishing comprises establishing presence of one or more sequences selected from the group consisting of SEQ ID NO: 1, SEQ ID NO: 2, SEQ ID NO: 6, and SEQ ID NO: 7 in a genome of the lettuce plant or the seed,
or
wherein the step of establishing comprises establishing presence of one or more sequences selected from the group consisting of a combination of SEQ ID NOS: 2 and 4, a combination of SEQ ID NOS: 2 and 5, and a combination of SEQ ID NOS: 2, 4, and 5 in a genome of the lettuce plant or the seed.

14. A method for providing a lettuce plant that is resistant to downy mildew, comprising the steps of:
a) crossing a lettuce plant comprising the resistance gene according to claim 9 with a lettuce plant that is susceptible to downy mildew and does not comprise said resistance gene,
b) optionally, selfing the plant obtained in step a) for at least one time,
c) selecting the plants that are resistant to downy mildew.

15. A method, comprising using a gene construct or a plasmid for introducing a resistance gene into the genome of a plant or plant cell and providing broad spectrum resistance to downy mildew caused by one or more of B. lactucae selected from the group of race Bl:1-37EU and/or BL1-9US, wherein the gene construct comprises the resistance gene according to claim 9 operably linked to expression providing sequences in said plant.

16. The lettuce plant according to claim 1, wherein said lettuce plant does not comprise SEQ ID NO: 6 and/or SEQ ID NO: 7, and wherein said lettuce plant provides an average seed production of at least 9 g of seed per lettuce plant or at least 14 g of seed per lettuce plant.

17. The lettuce plant according to claim 1, wherein the lettuce plant is Lactuca sativa.

18. The lettuce plant according to claim 9, wherein the resistance gene provides resistance to Bremia lactucae races Bl:1-37EU and Bl:1-9US in the lettuce plant.

19. The method of claim 12, wherein the step of establishing comprises establishing presence of one or more sequences selected from the group consisting of SEQ ID NO: 2, a combination of SEQ ID NOS: 2 and 6, and a combination of SEQ ID NOS: 2 and 7 in a genome of the lettuce plant or the seed.

20. The method of claim 12, wherein the step of establishing comprises establishing presence of the combination of SEQ ID NOS: 2, 4, and 5 in a genome of the lettuce plant or the seed.
